(19) United States
(12) Patent Application Publication
(10) Pub. No.: US 2026/0007818 A1
(43) Pub. Date: Jan. 8, 2026

(54) Nutritional Infusion Tubing

(71) Applicants: Medcaptain Medical Technology Co., Ltd., Shenzhen (CN); Medcaptain Life Science Co.,Ltd., Shenzhen (CN)

(72) Inventors: Xiang LI, Shenzhen (CN); Pan HU, Shenzhen (CN); Zhao WU, Shenzhen (CN); Zhiwu DENG, Shenzhen (CN); Haoke HUANG, Shenzhen (CN)

(73) Assignees: Medcaptain Medical Technology Co., Ltd., Shenzhen (CN); Medcaptain Life Science Co.,Ltd., Shenzhen (CN)

(21) Appl. No.: 19/258,610

(22) Filed: Jul. 2, 2025

(30) Foreign Application Priority Data
Jul. 4, 2024 (CN) 202410893456.9
Jul. 4, 2024 (CN) 202410894805.9
Jul. 4, 2024 (CN) 202421580829.9
Jul. 4, 2024 (CN) 202421581830.3

(51) Int. Cl. A61M 5/142; A61J 15/00

(52) U.S. Cl. CPC A61M 5/14212; A61J 15/0076

(57) ABSTRACT

A nutritional infusion tubing assembly includes a peristaltic tube, an input tube connector, an output tube connector, infusion tubes, and a support plate. The support plate is provided with a guide channel that guides the movement of the support plate. The input tube connector is connected to the peristaltic tube and an infusion tube, and the output tube connector connects the peristaltic tube to another infusion tube. A section of the peristaltic tube close to the input end and a section of the peristaltic tube close to the output end are subjected to an outward expansion force from the support plate, the input tube connector, or the independent expansion member. The internal structures of the nutritional infusion tubing assembly can provide expansion force sufficient to enable one-handed nutritional tubing installation. Additionally, due to the guiding effect of the guide channel, the installation process is highly reliable.

### REFERENCE TO RELATED APPLICATIONS

The invention claims priority of Chinese Patent Application No. 202410893456.9, entitled “Method of Assembling Nutritional infusion tubing assembly for Nutritional Infusion Device and Nutritional infusion system” filed with the China National Intellectual Property Administration on Jul. 4, 2024; Chinese Patent Application No. 202421581830.3, entitled “Nutritional infusion tubing assembly” filed with the China National Intellectual Property Administration on Jul. 4, 2024, Chinese Patent Application No. 202410894805.9, entitled “Nutritional infusion tubing assembly” filed with the China National Intellectual Property Administration on Jul. 4, 2024; and Chinese Patent Application No. 202421580829.9, entitled “Nutritional infusion tubing assembly” filed with the China National Intellectual Property Administration on Jul. 4, 2024, which are all incorporated herein by reference in their entirety.

### TECHNICAL FIELD

This invention generally relates to medical devices and more particularly to nutritional infusion.

### BACKGROUND ART

A nutritional infusion tubing assembly is usually included in a nutritional infusion device for conveniently delivering water, nutritional solutions, and pre-prepared meal emulsions of specific concentrations to patients. As a single-use consumable, the smaller the size of the nutritional infusion tubing assembly, the more convenient it is for patients to use, carry, and replace. However, when the nutritional infusion tubing is smaller in size, the installation process onto the nutritional infusion device requires coordinated operation with both hands for successful assembly, making the operation more inconvenient and the assembly procedure more complicated.

### SUMMARY OF THE INVENTION

Embodiments of this invention provide nutritional infusion tubing assembly that enables a compact design while allowing the operator to use it with one hand, making it more convenient for the operator to use. It also improves the reliability and convenience of the assembly process when installing the nutritional infusion tubing assembly into the nutritional infusion device, enabling a faster and simpler installation process.

In a first aspect, embodiments of this invention provide an nutritional infusion tubing assembly, comprising: a peristaltic tube, an input tube connector, an output tube connector, infusion tubes, and a support plate;
wherein the support plate is provided with a guide channel and two mounting grooves, with the two mounting grooves located on opposite sides of the guide channel, respectively;
the guide channel is configured to guide movement of the support plate;
an input end of the peristaltic tube is connected to one end of the input tube connector, the other end of the input tube connector is connected to at least one of the infusion tubes, and the input tube connector is located in one of the mounting grooves; an output end of the peristaltic tube is connected to one end of the output tube connector, the other end of the output tube connector is connected to another one of the infusion tubes, and the output tube connector is located within the other mounting groove; between the input end and the output end, the peristaltic tube further comprises an annular portion suspended relative to the support plate or an independent expansion member;
a section of the peristaltic tube between the input end and a starting point of the adjacent annular portion is subjected to an outward expansion force from the support plate, the input tube connector, or the independent expansion member;
a section of the peristaltic tube between the output end and a starting point of the adjacent annular portion is subjected to the outward expansion force from the support plate, the output tube connector, or the independent expansion member;
the outward expansion force has a component along a direction perpendicular to an extension direction of the guide channel, thereby increasing a distance between the two starting points of the annular portion along the direction perpendicular to the extension direction of the guide channel.

In a second aspect, embodiments of this invention provide a nutritional infusion tubing assembly comprising a peristaltic tube, an input tube connector, an output tube connector, infusion tubes, and a support plate, wherein the input tube connector and the output tube connector are both fixed to the support plate, one end of the input tube connector is connected to an input end of the peristaltic tube, and the other end of the input tube connector is connected to at least one infusion tube; one end of the output tube connector is connected to an output end of the peristaltic tube, and the other end of the output tube connector is connected to another infusion tube; the support plate comprises a plate body and two support members, wherein the input tube connector and the output tube connector are both fixed to the plate body, and the two support members are spaced apart on the same side of the plate body or the two support members are arranged on the corresponding opposite sides of the plate body; one support member abuts the input end of the peristaltic tube, and the other support member abuts the output end of the peristaltic tube, to prevent at least part of the input end of the peristaltic tube and at least part of the output end of the peristaltic tube from approaching each other in a direction toward a centerline of the plate body.

In a third aspect, embodiments of this invention provide a nutritional infusion tubing assembly, comprising: a peristaltic tube, an input tube connector, an output tube connector, infusion tubes, and a support plate;
wherein the support plate is provided with a guide channel and two mounting grooves, with the two mounting grooves located on opposite sides of the guide channel, respectively;
the guide channel is configured to guide movement of the support plate;
an input end of the peristaltic tube is connected to one end of the input tube connector, the other end of the input tube connector is connected to at least one of the infusion tubes, and the input tube connector is located in one of the mounting grooves; an output end of the peristaltic tube is connected to one end of the output tube connector, the other end of the output tube connector is connected to another one of the infusion tubes, and the output tube connector is located within the other mounting groove; between the input end and the output end, the peristaltic tube further comprises an annular portion suspended relative to the support plate or an independent expansion member; and
the maximum spacing of the annular portion in the extension direction of the guide channel is greater than or equal to 33 mm.

In a fourth aspect, embodiments of this invention provide a nutritional infusion tubing assembly, comprising: a peristaltic tube, an input tube connector, an output tube connector, infusion tubes, and a support plate;
wherein the support plate is provided with a guide channel and two mounting grooves, with the two mounting grooves located on opposite sides of the guide channel, respectively;
the guide channel is configured to guide movement of the support plate;
an input end of the peristaltic tube is connected to one end of the input tube connector, the other end of the input tube connector is connected to at least one of the infusion tubes, and the input tube connector is located in one of the mounting grooves; an output end of the peristaltic tube is connected to one end of the output tube connector, the other end of the output tube connector is connected to another one of the infusion tubes, and the output tube connector is located within the other mounting groove; between the input end and the output end, the peristaltic tube further comprises an annular portion suspended relative to the support plate or an independent expansion member;
the support plate comprises a plate body and two support members, with the two support members spaced apart on a bottom side of the plate body and extending toward the annular portion; and
one of the support members abuts the starting point in one end of the annular portion, and the other support member abuts the starting point of the other end in the annular portion, thereby increasing the distance between the two starting points of the annular portion along the direction perpendicular to the guide channel.

In the nutritional infusion tubing assembly provided by the embodiments of this invention, when the operator installs the nutritional infusion tubing assembly, the guide channel can serve as a guide and positioning device. Furthermore, when the nutritional infusion tubing assembly is in its normal state (i.e., when the nutritional infusion tubing assembly is not subjected to external forces from the operator or other external structural components), the structural components within the nutritional infusion tubing assembly, such as the local portion of the support plate, the input tube connector, the output tube connector, or independent expansion members, can exert an outward expansion force on the peristaltic tube, thereby increasing the distance between the two starting points of the annular portion of the peristaltic tube along the direction perpendicular to the guide channel. Such a design facilitates the operator's ability to easily slide the annular portion using only one hand onto the roller of the nutritional infusion device in the installation direction of the guide channel, thereby simplifying the assembly of the guide channel. This reduces the number of steps required for installation, lowers the difficulty of mounting the nutritional infusion tubing assembly onto the nutritional infusion device, and also contributes to minimizing the lateral dimensions and overall volume of both the nutritional infusion tubing assembly and its corresponding nutritional infusion device.

### BRIEF DESCRIPTION OF THE DRAWINGS

FIG. 1 is a structural schematic diagram of the nutritional infusion system according to some embodiments of this invention.

FIG. 2 is a structural schematic diagram of the nutritional infusion system shown in FIG. 1 in another state.

FIG. 3 is a schematic diagram of an installation environment of a nutritional infusion tubing assembly according to some embodiments of this invention.

FIG. 4 is a structural schematic diagram of the nutritional infusion tubing assembly shown in FIG. 2 according to some embodiments of this invention.

FIG. 5 is a structural schematic diagram of the nutritional infusion tubing assembly shown in FIG. 4 from another perspective.

FIGS. 6A and 6B together form an exploded view of the nutritional infusion tubing assembly shown in FIG. 5.

FIGS. 7A and 7B are schematic diagrams of a partial process of installing the nutritional infusion tubing assembly into the nutritional infusion device according to an embodiment of this invention.

FIG. 8 is a structural schematic diagram of the support plate shown in FIG. 6 from another perspective.

FIG. 9 is a schematic diagram of the nutritional infusion tubing assembly during the installation process according to an embodiment of this invention.

FIG. 10 is a structural schematic diagram of another nutritional infusion tubing assembly according to an embodiment of this invention.

### DETAILED DESCRIPTION

The embodiments of this invention are described below in connection with the accompanying drawings of the embodiments of this invention. The following is a listing of the various parts depicted in the drawings, along with their reference numbers:
1: Nutritional infusion system, which includes the nutritional infusion tubing assembly 10 and the nutritional infusion device 20
10: Nutritional infusion tubing assembly
11: Peristaltic tube
111: Input section
111a: Input end
112: Output section
112a: Output end
113: Annular portion
113a: First starting point of annular portion 113
113b: Second starting point of annular portion 113
12: Input tube connector
121: Direction: changing groove
122: First end
123: Second end
13: Output tube connector
131: Third end
132: Fourth end
14: Infusion tube
141: Input tube
142: Output tube
15: Support plate
151: Plate body
151a: Top surface of support plate 15
151b: Bottom surface of support plate 15
151c: Slope
152: Protrusion
153: Locking hole
154: Support member
1541: Connection portion
1542: Bending portion
1543: support portion
1543a: Main body portion
1543b: First blocking plate
1543c: Second blocking plate
155: Mounting groove
155a: Mounting port
156: Pressing member
16: In: place identification element
20: Nutritional infusion device
201: Bottom surface of nutritional infusion device 20
21: Housing
211: Mounting surface
211a: First mounting surface
211b: Second mounting surface
211c: Third mounting surface
22: Pump door
23: Mounting space
23a: First mounting space
23b: Second mounting space
23c: Third mounting space
24: Driving assembly
241: Roller
25: Tube groove
251: Ultrasonic bubble sensor
252: Pressure sensor
26: Fixing assembly
27: Locking member
28: Switching member
30: Guide rail
301: First guide rail
302: Second guide rail
303: Third guide rail
31: Guide channel
311: First guide channel
312: Second guide channel.

In the description of the embodiments of this invention, it is to be noted that, unless otherwise expressly provided and limited, the terms “mount” and “connect” are to be broadly construed. For example, “connected” may be detachably connected or non-detachably connected, directly connected or indirectly connected through an intermediate medium. “Plurality” means at least two.

Orientation terms referred to in embodiments of this invention, such as “up”, “inner”, “outer”, “top” “bottom”, “side” are only orientations with reference to the accompanying drawings. Accordingly, the orientation terms used are intended to better and more clearly illustrate and understand the embodiments of this invention, and are not intended to indicate or imply that the device or element referred to must have a particular orientation, be constructed and operated in a particular orientation, and therefore are not to be construed as a limitation of the embodiments of this invention.

In the embodiments of this invention, the specified relative positional relationships (e.g., parallel, perpendicular) are not absolute, strict limitations; rather, minor deviations are permitted, including being approximately parallel or approximately perpendicular. For example, ‘A is parallel to B’ means that A and B are either perfectly parallel or approximately parallel, with an included angle between 0 and 10 degrees. Similarly, ‘A is perpendicular to B’ indicates that A and B are either precisely perpendicular or approximately perpendicular, with an included angle ranging from 80 to 100 degrees

In the embodiments of this invention, the terms “first” and “second” are used for descriptive purposes only and are not to be understood as indicating or implying relative importance or implicitly specifying the number of technical features indicated. As a result, the feature defined with “first” or “second” may expressly or implicitly include one or more such features. In this regard, the one-piece structural member means that one of the parts of the structural member is connected to the other part during the process of forming the other part, and there is no need to connect the two parts together by means of further processing (e.g., adhesive bonding, welding, snap tab connection, screw connection).

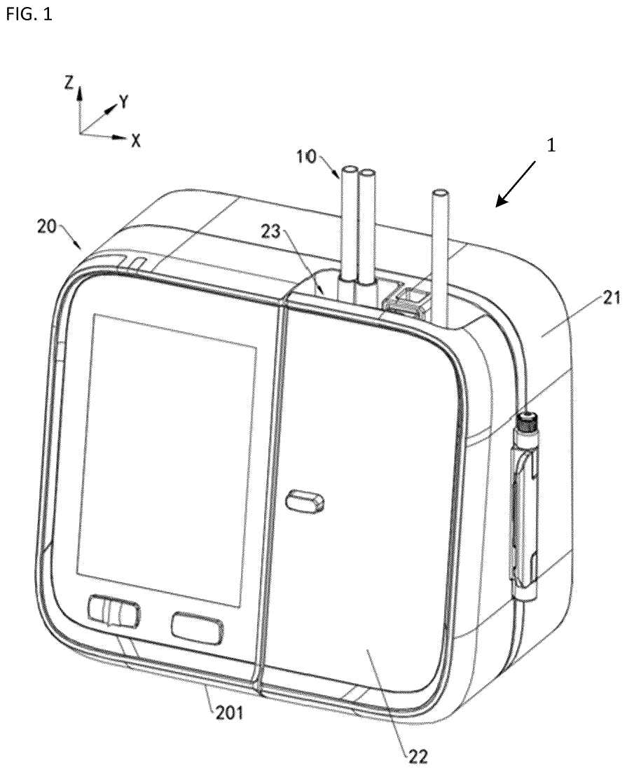

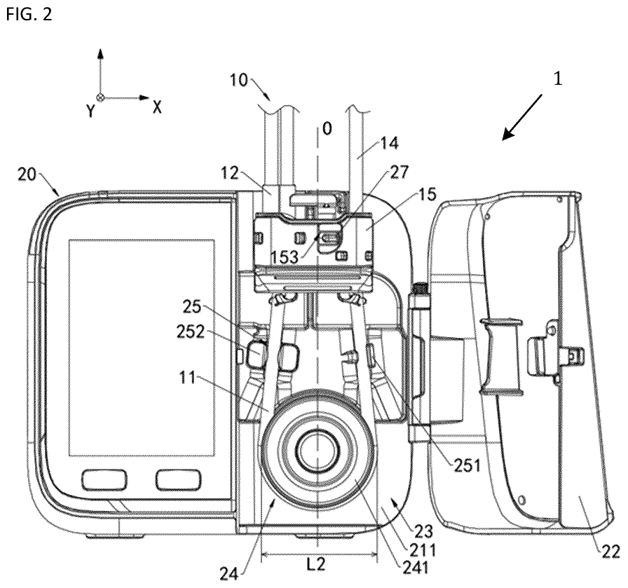

See FIG. 1 and FIG. 2, of which FIG. 1 is a structural schematic diagram of the nutritional infusion system according to some embodiments of this invention, and FIG. 2 is a structural schematic diagram of the nutritional infusion system shown in FIG. 1 in another state.

In some embodiments, the nutritional infusion system 1 may include nutritional infusion tubing assembly 10 and a nutritional infusion device 20, and the nutritional infusion tubing assembly 10 may be detachably mounted on the nutritional infusion device 20. In clinical practice, the nutritional infusion system 1 is generally utilized to deliver water, nutrient solution, and a pre-prepared food emulsion of a certain concentration to the patient, thereby providing nutritional support. The nutritional infusion device 20 may provide a driving force for the fluid within the nutritional infusion tubing assembly 10 to push and deliver the fluid within the nutritional infusion tubing assembly 10 to the patient.

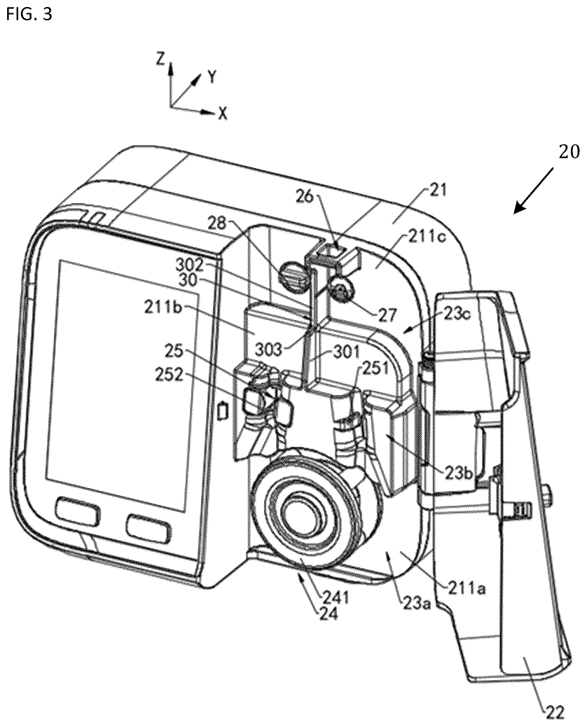

See FIG. 2 and FIG. 3, of which FIG. 3 is a schematic diagram of an installation environment of the nutritional infusion tubing assembly according to some embodiments of this invention.

In some embodiments, the nutritional infusion device 20 may include a housing 21 and a pump door 22, with the pump door 22 rotatably connected to one side of the housing 21. For example, the housing 21 may be made of materials such as plastic or silicone to provide structural support for the nutritional infusion device 20. The pump door 22 may be hinged to the housing 21 via a door hinge. In other embodiments, the pump door 22 may also be detachably connected to the housing 21 and reattached to the housing 21 when needed, or the pump door 22 may be slidably connected to the housing 21. This invention does not require any particular connection method between the pump door 22 and the housing 21. In some embodiments, for example, the nutritional infusion device 20 may not even include the pump door 22 at all.

For example, the nutritional infusion device 20 may have a mounting space 23, which may be formed by a portion of the outer surface of the housing 21 recessing toward the interior of the housing 21. The mounting space 23 may be configured to accommodate other structural components within the nutritional infusion system 1 (e.g., the nutritional infusion tubing assembly 10). During the process of opening the pump door 22 relative to the housing 21, the structural components installed within the mounting space 23 are exposed, facilitating the operator's installation or replacement of the structural components within the mounting space 23. When the pump door 22 is closed relative to the housing 21, the pump door 22 can cover the mounting space 23 to protect the structural components installed within the mounting space 23, preventing external contaminants such as water or dust from affecting the service life of the structural components within the mounting space 23. As shown in FIG. 1, the pump door 22 and the housing 21 can together form the outer contour of the nutritional infusion system 1. When the pump door 22 is closed relative to the housing 21, it restores the overall shape of the nutritional infusion system 1. The outer surface of the nutritional infusion system 1 can be considered approximately planar, ensuring the aesthetic appeal of the product.

As an example, as shown in FIG. 1, the nutritional infusion device 20 may be mounted on a support base such as a desktop or support frame. The nutritional infusion device 20 may be provided with a bottom surface 201 that may be configured to contact the support base such as the desktop or support frame. In some examples, the bottom surface 201 of the nutritional infusion device 20 may also be equipped with a flexible support pad to prevent direct contact between the nutritional infusion device 20 and the support base, thereby increasing the friction between the nutritional infusion device 20 and the support base, ensuring the stability of the nutritional infusion device 20, and reducing wear or scratches on the bottom surface 201 of the nutritional infusion device 20. The side on which the bottom surface 201 of the nutritional infusion device 20 is located is defined as the bottom side of the nutritional infusion device 20, and the top side of the nutritional infusion device 20 is opposite the bottom side.

It should be understood that, for the sake of convenience in description, in the description here of embodiments of this invention, the nutritional infusion device 20 shown in FIG. 1 is defined as being in an upright state, and the nutritional infusion device 20 shown in FIG. 1 is defined as having an X-axis direction, the Y-axis direction, and the Z-axis direction. The X-axis direction may be parallel to the length direction of the nutritional infusion device 20, the Z-axis direction may be parallel to the height direction of the nutritional infusion device 20, and the Y-axis direction may be parallel to the width direction of the nutritional infusion device 20. Any two of the X-axis direction, the Y-axis direction, and the Z-axis direction are mutually perpendicular. In other embodiments, the nutritional infusion device 20 may also be connected to the support base such as a table or support frame in a lying, suspended, or other posture. The coordinate system used to understand the various relative positions of the nutritional infusion device 20 may be chosen however preferred . . .

In some embodiments, the nutritional infusion device 20 may further include a driving assembly 24, which may include a roller 241. The roller 241 may be mounted within the mounting space 23 and be rotatably connected to the housing 21. For example, the housing 21 may be provided with a mounting surface 211, allowing the operator to face the mounting surface 211 of the nutritional infusion device 20 and manipulate the structural components within the mounting space 23. In the embodiments of this invention, the roller 241 may protrude relative to the mounting surface 211 and be positioned close to the bottom side of the nutritional infusion device 20. In other embodiments, the roller 241 may be positioned close to the top side of the nutritional infusion device 20 or at the middle position of the housing 21. This invention does not impose any restrictions in this regard.

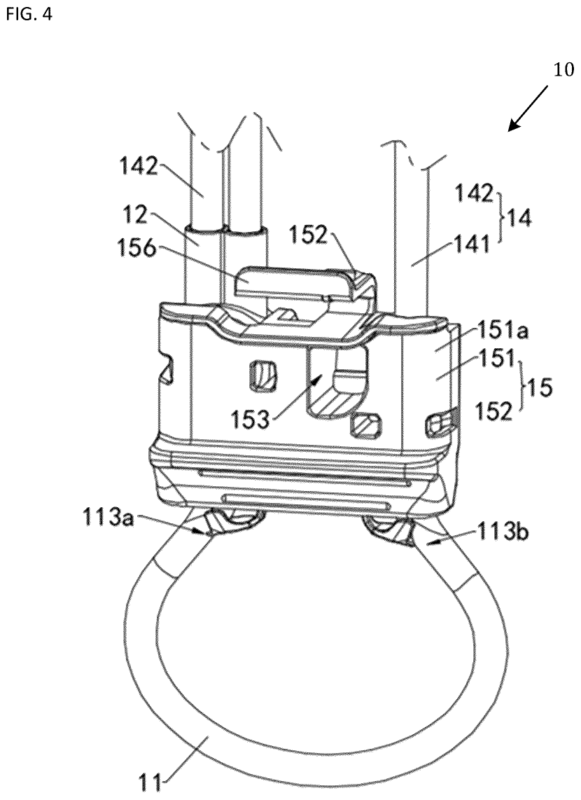

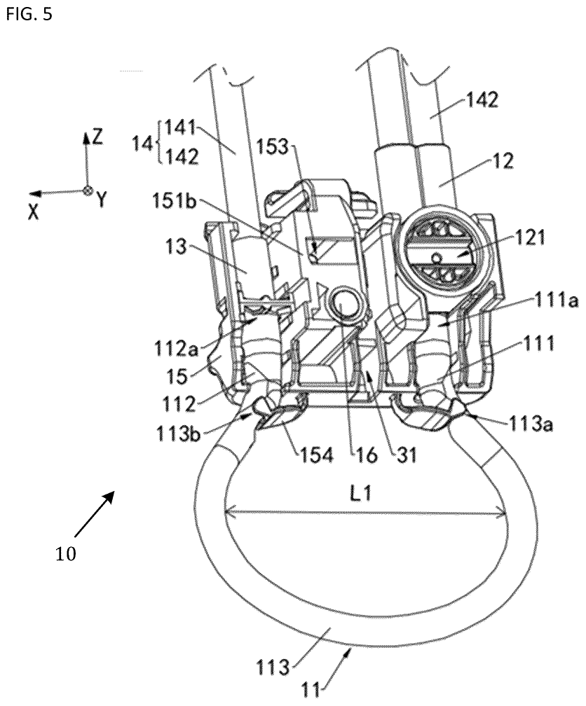

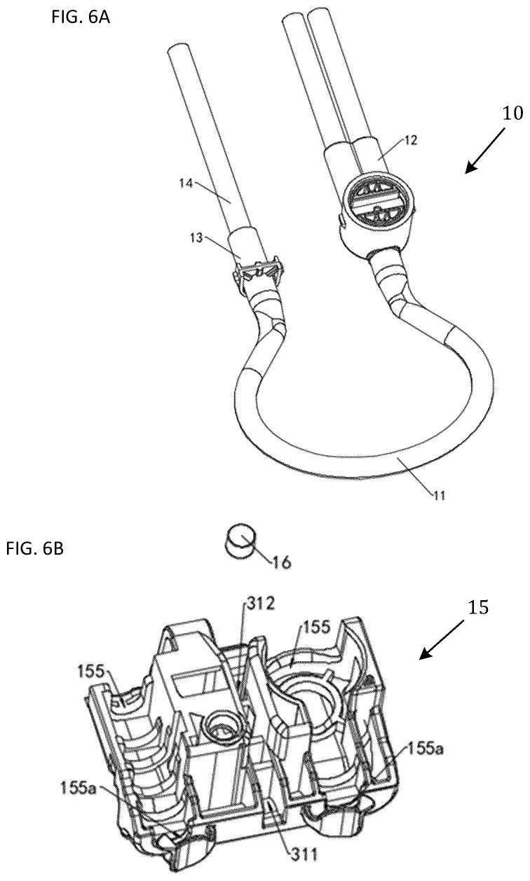

See FIGS. 4, 5, 6A and 6B, of which FIG. 4 is a structural schematic diagram of the nutritional infusion tubing assembly shown in FIG. 2 according to some embodiments of this invention, FIG. 5 is a structural schematic diagram of the nutritional infusion tubing assembly shown in FIG. 4 from another perspective, and FIGS. 6A and 6B show an exploded view of the nutritional infusion tubing assembly shown in FIG. 5.

In some embodiments, the nutritional infusion tubing assembly 10 may include a peristaltic tube 11, an input tube connector 12, an output tube connector 13, and one or more infusion tubes 14. One end of the input tube connector 12 is connected to the input end 111a of the peristaltic tube 11, and the other end of the input tube connector 12 is connected to one infusion tube 14. One end of the output tube connector 13 is connected to the output end 112a of the peristaltic tube 11, and the other end of the output tube connector 13 is connected to another infusion tube 14. By connecting the peristaltic tube 11, the input tube connector 12, the output tube connector 13, and the infusion tubes 14, the tubing components within the nutritional infusion tubing assembly 10 can be assembled into an integrated structure, enhancing the modularity of the internal structure of the nutritional infusion tubing assembly 10 and facilitating the ease of moving and installing the nutritional infusion tubing assembly 10. The peristaltic tube 11 may be approximately shaped like a “water droplet.” The input end 111a and the output end 112a of the peristaltic tube 11 represent the openings at both ends of the peristaltic tube 11, through which the peristaltic tube 11 can connect to the external space.

As an example, the peristaltic tube 11 may be made of materials such as silicone, which has high electrical heat resistance, insulation, reliability, bendability, and flexibility. In embodiments of this invention, the peristaltic tube 11 may be directly looped around the input tube connector 12 and/or the output tube connector 13 using its elasticity, or it may be connected to the input tube connector 12 and/or the output tube connector 13 by means of adhesive bonding, ultrasonic welding, etc. In other embodiments, the peristaltic tube 11 and the input tube connector 12 and/or the output tube connector 13 may be integrated into a single structure. In some embodiments, the input tube connector 12 and/or the output tube connector 13 may be omitted from the nutritional infusion tubing assembly 10, one end of the peristaltic tube 11 may be directly connected to one infusion tube 14, and the other end of the peristaltic tube 11 may be directly connected to another infusion tube 14.

For example, the number of infusion tubes 14 may be two, three, or more; the infusion tubes 52 serve as a fluid delivery path. The plurality of infusion tubes 14 may include an output tube 141 and at least one input tube 142. The plurality of input tubes 142 may be used for filling different fluids such as nutrient fluid, cleaning fluid, etc., into the peristaltic tube 11, respectively, which are then transported to the patient through the output tube 141. The infusion tubes 14 may be made from synthetic materials such as polyvinyl chloride (PVC) with additives or thermoplastic polyurethane elastomer rubber (TPU), or any other suitable material preferred by the manufacturer.

In some examples, the input tube connector 12 may be a valve mechanism such as a two-way valve with one inlet and one outlet or a three-way valve with two inlets and one outlet, whereby the position of the valve core in the control valve is controlled to control the connectivity of the passage between the infusion tubes 14 and the peristaltic tube 11. For example, the infusion tubes 14 may be connected to one of the inlets and the outlet of the peristaltic tube 11. As another example, the infusion tubes 14 may be connected to the other inlet and the outlet of the peristaltic tube 11. Alternatively, the passage between the infusion tubes 14 and the peristaltic tube 11 may be blocked, so as to realize on-off switching of the fluid pathway in the nutritional infusion tubing assembly 10—such a valve mechanism is more flexible and reliable, which helps to improve the reliability of the nutritional infusion tubing assembly 10 and the nutritional infusion system 1. Before the nutritional infusion tubing assembly 10 is mounted onto the nutritional infusion device 20 (as shown in FIG. 2), the nutritional infusion tubing assembly 10 may be in a blocked state. After the nutritional infusion tubing assembly 10 is mounted to the nutritional infusion device 20, the three-way/two-way valves may be controlled by rotating the angle of the valve core to connect to the corresponding tube according to the setting to realize the desired nutritional fluid feeding. After stopping feeding, the three-way/two-way valve core may be reset to a blocking state by rotating the control valve core to avoid uncontrolled free flow in the nutritional infusion tubing assembly 10 after it is removed from the nutritional infusion device 20, so as to improve the reliability and safety of the nutritional infusion system 1. In other embodiments, the input tube connector 12 may have a different structure.

In the embodiment of the invention shown in FIG. 2, the tubing of the nutritional infusion tubing assembly 10 can be looped around the outer periphery of the roller 241, such that the roller 241 can exert a certain pressure on the tubing. For example, when the nutritional infusion tubing assembly 10 is properly installed, the peristaltic tube 11 can contact at least part of the roller 241. When the roller 241 rotates, the peristaltic tube 11 is stretched, and the roller 241 can exert a pushing force on the fluid inside the peristaltic tube 11, pushing the fluid inside the peristaltic tube 11 to flow along the tubing, thereby delivering the nutrients entering the peristaltic tube 11 to the patient. When the roller 241 stops rotating, the pressure exerted by roller 241 on the tubing of peristaltic tube 11 is significant, effectively blocking the fluid pathway within the tubing of peristaltic tube 11, causing the fluid within the tubing to cease flowing forward and thus halting the delivery of nutrients to the patient.

In some embodiments, the nutritional infusion tubing assembly 10 may further include a support plate 15, with the input tube connector 12 and output tube connector 13 both fixed to the support plate 15. By connecting the input tube connector 12 and output tube connector 13 via the support plate 15, the support plate 15 can be configured to fix the relative positions of the input tube connector 12 and output tube connector 13. This arrangement effectively maintains the structural configuration of the nutritional infusion tube 10, providing the flexible tubing with a defined shape and stable positioning for facilitated installation. Furthermore, it enhances the structural integration of the nutritional infusion tube 10 and significantly reduces the operational difficulty during handling and repositioning of the tube.

As shown in FIGS. 5, 6A and 6B, for example, the support plate 15 may have two mounting grooves 155, with the input tube connector 12 and the output tube connector 13 respectively fixed within the two mounting grooves 155. The input tube connector 12 and/or the output tube connector 13 may be fixed in the mounting grooves 155 through snap-fit connection, adhesive bonding, or similar fastening methods. The mounting grooves 155 can serve to limit and position the input tube connector 12 and the output tube connector 13 located within them. By positioning the input tube connector 12 and the output tube connector 13 within the mounting grooves 155, the mounting grooves 155 also provide support for the input tube connector 12 and the output tube connector 13, thereby enhancing the structural strength of the connection points between the input tube connector 12, the peristaltic tube 11, the output tube connector 13, and the infusion tubes 14, and improving the connection reliability of the nutritional infusion tubing assembly 10. The peristaltic tube 11 may include an annular portion 113 (as shown in FIG. 5), and the annular portion 113 may be suspended relative to the support plate 15. It can be understood that the annular portion 113 being suspended relative to the support plate 15 means that the annular portion 113 is not subjected to the force of the support plate 15, or is not within the range of the force exerted by the support plate 15. For example, the support plate 15 has one support point at each end of the peristaltic tube 11, such that the annular portion is connected between the two support points of the support plate 15 and the peristaltic tube 11, and is located on the side of the two support points away from the support plate 15. These two support points can also be considered as the starting points of the annular portion 113, and the annular portion 113 may include an input end point 113a and an output end point 113b. In the embodiment of this invention, the section of the peristaltic tube 11 between the input end 111a and the input end point of the adjacent annular portion 113 (the first end point 113a of the annular portion 113) is referred to as the input section 111 of the peristaltic tube 11 (as shown in FIG. 5), and a section of the peristaltic tube 11 between the output end 112a and the output end point of the adjacent annular portion 113 (the second end point 113b) is referred to as the output section 112 of the peristaltic tube 11 (as shown in FIG. 5).

In some embodiments, the nutritional infusion tubing assembly 10 may further include an independent expansion member (not shown in the figures). The independent expansion member refers to an expansion member that has no connection relationship with structural components such as the support plate 15, the input tube connector 12, and the output tube connector 13. The annular portion 113 may also be suspended relative to the independent expansion member. In this case, the two ends of the expansion member may be abut the peristaltic tube 11, the annular portion 113 may be located on the portion of the expansion member facing away from the support plate 15, and the two end points of the annular portion 113 may be the two contact points of the expansion member on the peristaltic tube 11. In other words, the annular portion 113 can be understood as a section of the peristaltic tube 11 that is not subjected to the forces exerted by the expansion member and the support plate 15, or is outside the range of influence of the forces exerted by the expansion member and the support plate 15. The following description will exemplify the configuration of the nutritional infusion tubing assembly 10 by taking the annular portion 113 suspended relative to the support plate 15 as an example. When the annular portion 113 is suspended relative to the support plate, the relevant configuration of the nutritional infusion tubing assembly 10 may be adaptively modified with reference to the following content.

In particular, the arrangement direction of the two mounting grooves 155 may be parallel to the arrangement direction of the input tube connector 12 and the output tube connector 13. In the embodiment of this invention, the input tube connector 12 and the output tube connector 13 may be arranged along the X-axis direction, i.e., the two mounting grooves 155 may be arranged along the X-axis direction. In other embodiments, the arrangement direction of the two mounting grooves 155 may intersect or be perpendicular to the arrangement direction of the input tube connector 12 and the output tube connector 13, or have some other relative orientation.

The support plate 15 may have a top surface 151a (as shown in FIG. 4) and a bottom surface 151b (as shown in FIG. 5), which are located on opposite sides of the support plate 15 in the Y-axis direction. The bottom surface 151b of the support plate 15 is closer to the mounting surface 211 than the top surface 151a of the support plate 15. The mounting groove 155 can be considered as being formed by a portion of the bottom surface 151b recessing toward the top surface 151a.

As shown in FIGS. 2 and 3, in some embodiments, the nutritional infusion device 20 may have two tube grooves 25. After the nutritional infusion tubing assembly 10 is installed in the nutritional infusion device 20, the two sides of the annular portion 113 may be fixed in the two tube grooves 25, respectively. By providing the tube grooves 25 within the nutritional infusion device 20, the tube grooves 25 can limit the extension direction of the peristaltic tube 11, thereby facilitating smoother looping of the nutritional infusion tubing assembly 10 around the outer periphery of the roller 241. Furthermore, the provision of the tube grooves 25 to fix the relative position between the nutritional infusion tubing assembly 10 and the nutritional infusion device 20 can effectively prevent issues such as dislodgement of the nutritional infusion tubing assembly 10 when external factors (e.g., accidental operator contact with the nutritional infusion system 1) occur.

For example, the walls of the tube grooves 25 may be provided with an ultrasonic bubble sensor 251. When the peristaltic tube 11 is slotted in the tube grooves 25, the ultrasonic bubble sensor 251 can then emit and receive ultrasonic waves to determine the presence of bubbles based on differences in their reflection and propagation, thereby enabling detection of bubbles within the peristaltic tube 11. This enhances the safety and reliability of the nutritional infusion system 1.

As an example, the nutritional infusion device 20 may also include other detection devices such as a pressure sensor 252 to monitor parameters such as the blockage pressure in the peristaltic tube 11, thereby enabling monitoring of the normal and smooth operation of the nutritional infusion system 1. This allows the operator to promptly understand and control the operational status of the nutritional infusion system 1. This invention does not impose any limitations in this regard. In the embodiments of this invention, the two tube grooves 25 may each be equipped with an ultrasonic bubble sensor 251 and a pressure sensor 252. FIGS. 2 and 3 merely illustrate the positions of the ultrasonic bubble sensor 251 and the pressure sensor 252 for reference purposes, and the design may be adjusted according to actual requirements.

For example, the tube grooves 25 may be positioned on a side of the roller 241 facing the top side of the nutritional infusion device 20. The distance between the ends of the two tube grooves 25 closer to the roller 241 may be greater than the distance between the ends of the two tube grooves 25 and the roller 241. The ends of the two tube grooves 25 closer to the roller 241 may form an approximate “flared opening structure” shape. By configuring the profile of the tube grooves 25, the groove's superior forming capability is utilized to maintain the nutritional infusion tubing assembly 10 in a “teardrop-shaped” bending configuration. This design facilitates smoother looping of the peristaltic tube 11 around the roller 241. In other embodiments, the relative position of the tube grooves 25 and the roller 241 may also be flexibly set according to actual needs, and the tube grooves 25 may also be straight, curved, or have some other shape. Alternatively, in some other embodiments, the nutritional infusion device 20 may omit the tube grooves 25 altogether.

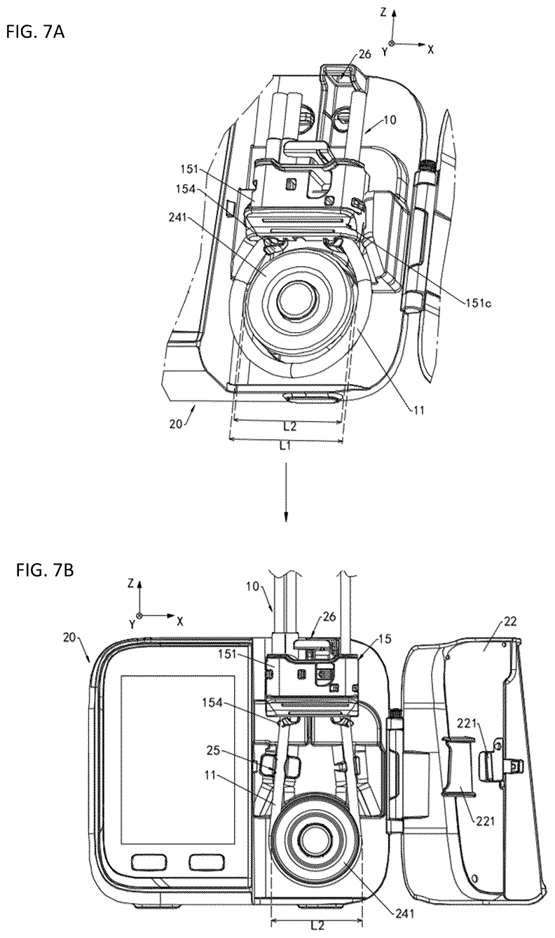

FIGS. 7A and 7B are, together, a schematic diagram of a partial process of installing the nutritional infusion tubing assembly 10 into the nutritional infusion device 20 according to an embodiment of this invention. FIG. 7A shows the state where the nutritional infusion tubing assembly 10 provided by the embodiment of this invention has just been installed on the roller 241, and FIG. 7B shows the state where the nutritional infusion tubing assembly 10 provided by the embodiment of this invention is fully installed.

The process of installing the nutritional infusion tubing assembly 10 on the nutritional infusion device 20 generally includes: looping the peristaltic tube 11 over the outer circumference of the roller 241; moving the support plate 15 to stretch the peristaltic tube 11, so that the peristaltic tube 11 can come into contact with at least part of the roller 241; and fixing the nutritional infusion tubing assembly 10 to the nutritional infusion device 20.

In the prior art, when the dimensions of whatever type of nutritional infusion tubing assembly is included are made smaller, the elastic force of the peristaltic tube is relatively large, and the space between the two ends of the peristaltic tube is relatively small. This requires the operator to use both hands in coordination to first overcome the relatively large elastic force of the peristaltic tube and then stretch it enough to be able to then install it over the roller. While moving some kind of support plate to stretch the peristaltic tube, the operator must then also grip the upper and lower ends of the support plate (in the embodiment of this invention, these are the two ends of the support plate along the Z-axis) and apply force. During the stretching process, the prior art's version of the support plate is suspended in the air, making it difficult for the operator to manipulate and apply force, and that support plate is also prone to deviating from its pre-set position, affecting the accuracy and reliability of the installation position of the nutritional infusion tubing assembly after it is installed on the nutritional infusion device. Additionally, during the process of fixing the nutritional infusion tubing assembly to the nutritional infusion device 20, the prior art support plate must be pressed down along a direction perpendicular to the roller (in the embodiment of this invention, this is also the Y-axis direction) to cause the support plate to engage the structural components on the nutritional infusion device designed to fix the support plate. However, pressing a support plate downward while it is suspended can cause it to fall vertically, potentially damaging it. As such, the assembly process of the prior art is cumbersome, requiring the operator to use both hands, making it inconvenient and unreliable.

In the prior art, when some form of a nutritional infusion tubing assembly is made smaller, the peristaltic tube has greater elasticity and the space between the two ends of the peristaltic tube is also smaller. This then requires the operator to use both hands to first overcome the greater elasticity of the peristaltic tube and then stretch it enough to then mount it onto the roller. Moreover, during the process of moving a support plate according to the prior art to stretch the peristaltic tube, it is necessary to grip the upper and lower ends of the support plate (i.e., the ends of the support plate in the Z-axis direction in the embodiments of this invention) to apply force. During the stretching process, the prior art support plate is suspended in the air, making it difficult for the operator to handle and apply force, and the support plate is prone to deviate from the preset position, affecting the accuracy and reliability of the installation position after the nutritional infusion tubing assembly is mounted on the nutritional infusion device. Additionally, during the process of securing the nutritional infusion tubing assembly to the nutritional infusion device, the prior art support plate is pressed downward in a direction perpendicular to the roller (i.e., the Y-axis direction in the embodiments of this invention) to cause it to engage the structural component on the nutritional infusion device designed to fix the support plate. However, pressing a suspended support plate downward to make it fall vertically may easily damage it. Thus, the assembly process in the prior art involves cumbersome steps, requires the operator to work with both hands, and is relatively inconvenient to operate with low reliability.

Therefore, as shown in FIGS. 2, 4, and 5, compared with the prior art, the embodiments of this invention provide an improved nutritional infusion tubing assembly 10. Compared with conventional structures, the nutritional infusion tubing assembly 10 features a modified design that enables one-handed operation for mounting onto the nutritional infusion device 20, with a simplified and easily achievable installation process.

As shown in FIGS. 5, 7A and 7B, in some embodiments, the section of the peristaltic tube 11 (i.e., the input section 111) between the input end 111a and the input end point 113a of the adjacent annular portion 113, is subjected to an outward expansion force from the support plate 15. The section of the peristaltic tube 11 (i.e., the output section 112) between the output end 112a and the output point 113b of the adjacent annular portion 113 is subjected to an outward expansion force from the support plate 15. This outward expansion force is used to increase the distance between the two starting points of the annular portion 113 in the direction perpendicular to the movement direction of the support plate 15. The direction of the outward expansion force may intersect or be perpendicular to the movement direction of the support plate 15. In the embodiments of this invention, the direction of the outward expansion force intersects or is parallel to the X-axis direction.

By arranging the internal structure of the nutritional infusion tubing assembly 10 as in this embodiment of the invention, the peristaltic tube 11 can be subjected to the outward expansion force, so that the nutritional infusion tubing assembly 10 has a stable shape before it is looped around the roller 241 (i.e., before the nutritional infusion tubing assembly 10 is in its normal state or before it is deformed by the force of the roller 241 or before the external force is applied by the operator), and the maximum spacing L1 of the peristaltic tube 11 in the first direction can be greater than or equal to the maximum spacing L2 between any two points on the roller 241; in other words, the “loop” of the peristaltic tube is at least as wide as the roller. It should be understood that the maximum spacing L1 of the peristaltic tube 11 in the first direction refers to the maximum spacing L1 of the peristaltic tube 11 in the first direction (as shown in FIG. 5). When the nutritional infusion tubing assembly 10 is in its normal state, the maximum distance L1 of the peristaltic tube 11 is greater than or equal to the maximum distance L2 between any two points on the roller 241 (as shown in FIG. 2). This configuration allows the peristaltic tube 11 to be effortlessly looped around the periphery of the roller 241, thereby eliminating the conventional requirement for users to overcome the elastic force of the peristaltic tube 11 and stretch it open before mounting. Consequently, the nutritional infusion tubing assembly 10 can be looped with only one hand around the outer circumference of the roller 241, significantly simplifying the assembly process of the nutritional infusion tubing assembly 10 onto the nutritional infusion device 20 and enhancing operator convenience.

The first direction may be parallel to the mounting surface 211 of the housing 21, for example, the first direction may be parallel to the X-axis direction. In other embodiments, the first direction may also be parallel to the Y-axis direction or Z-axis direction, or intersect with the X-axis direction and/or Y-axis direction and/or Z-axis direction, which is not limited in this invention. In the embodiments of this invention, the arrangement direction of the input tube connector 12 and the output tube connector 13 may be parallel to the X-axis direction, i.e., the first direction may be parallel to the arrangement direction of the input tube connector 12 and the output tube connector 13. In other embodiments, the arrangement direction of the input tube connector 12 and the output tube connector 13 may also intersect with the X-axis direction, which is not limited in the embodiments of this invention.

In the illustrated embodiment of this invention, the roller 241 may be approximately cylindrical in shape, and the maximum distance L2 between any two points on the roller 241 may also be understood as the length of the diameter of the roller 241. That is, the maximum distance L1 of the peristaltic tube 11 in the first direction may be greater than or equal to the diameter (maximum distance L2) of the roller 241 in the first direction, enabling one-handed looping of the undeformed peristaltic tube 11 around the outer circumference of the roller 241. In this circumstance, the plane of the peristaltic tube 11 remains parallel to the end face of the roller while maintaining coaxial alignment, with the connecting line between the roller's two maximum-spacing points parallel to the first direction (X-axis direction). The undeformed state refers to when no external force is applied by the operator, where the peristaltic tube 11 may exist in either a completely undeformed state or exhibit minimal deformation due to internal structural support within the nutritional infusion tubing assembly 10.

In one implementation of this invention, the diameter of the end face of the roller 241 was 33 mm. For example, the maximum spacing L1 of the peristaltic tube 11 in the first direction when the nutritional infusion tubing assembly 10 is in its normal state (which can also be considered as the maximum spacing of annular portion 113 in the direction perpendicular to the movement direction of the support plate 15) was, in that implementation, greater than or equal to 33 mm. In other embodiments, the maximum distance L2 between any two points on the roller may instead be other values such as 30 mm, 32 mm, 35 mm, 38 mm, etc., and the maximum distance L1 of the peristaltic tube 11 in the first direction when the nutritional infusion tubing assembly 10 is in its normal state may be set as needed.

The direction of the line connecting any two points on the roller 241 may also form an angle with the first direction. In the embodiments of this invention, that is, the line connecting the two points with the maximum spacing on the roller 241 may form an angle with the X-axis direction. For example, the cross-sectional shape of the surface of the roller 241 may also be elliptical, square, triangular, or other shapes. The maximum distance between any two points on the roller 241 may be the length of the major axis of the roller 241 or the length of the diagonal of the roller 241. When looping the peristaltic tube 11 around the outer circumference of the roller 241, the undeformed peristaltic tube 11 may initially contact a portion of the outer circumference of the roller 11. Using the contact point between the peristaltic tube 11 and the roller 241 as a force-applying point, slight deformation is induced by moving the support plate 15, enabling subsequent contact between the remaining portion of the peristaltic tube 11 and the other outer circumference of the roller 241. The deformation force required for plate movement is substantially less than the maximum one-handed operational force. Here, “moving the support plate 15” encompasses rotational or translational displacement at arbitrary angles to achieve proper alignment with mounting surface 211, such that either one edge of the support plate 15 in the Z-axis direction becomes parallel to the X-axis direction, or the arrangement direction of both edges of the support plate 15 in the X-axis direction becomes parallel to the X-axis direction.

As shown in FIGS. 5, 7A, 7B and 8, FIG. 8 is a structural schematic diagram of the support plate shown in FIGS. 6A and 6B from another perspective.

In some embodiments, the support plate 15 may include a plate body 151 and a support member 154, with the support member 154 positioned on a side of the plate body 151 facing toward the roller 241. The support member 154 may abut against the peristaltic tube 11 to prevent the input section 111 of the peristaltic tube 11 (as shown in FIG. 5) from approaching the output section 112 of the peristaltic tube 11 (as shown in FIG. 5), ensuring that the maximum distance L1 of the peristaltic tube 11 in the first direction (i.e., the X-axis direction) when the nutritional infusion tubing assembly 10 is in its normal state is greater than or equal to the maximum distance L2 between any two points of the roller 241. For example, the number of support members 154 may be two, with the two support members 154 spaced apart on the same side of the plate body 151. The two support members 154 can respectively abut the starting point of one end of the annular portion 113 and the starting point of the other end of the annular portion 113. In some embodiments of this invention, the abutment force exerted by the two support members 154 on the peristaltic tube 11, i.e., the outward expansion force on the peristaltic tube 11 exerted by the support plate 15, to increase the distance between the two starting points of the annular portion 113 in the direction perpendicular to the movement direction of the support plate 15 (i.e., the X-axis direction), so that the maximum spacing L1 of the peristaltic tube 11 in the first direction when the nutritional infusion tubing assembly 10 is in its normal state is greater than or equal to the maximum spacing L2 between any two points of the roller 241, making it convenient for the operator to hold the nutritional infusion tubing assembly 10 with one hand and easily loop it around the roller 241. In some embodiments, the two support members 154 may also be arranged on opposite sides of the plate body 151, for example, the two support members 154 may also be arranged on opposite sides of the plate body 151 in the X-axis direction, which is not limited in this invention.

In some embodiments, the plate body 151 and/or support members 154 may be made of materials such as acrylonitrile-butadiene-styrene (ABS) resin, which has high strength and toughness; manufacturers may, however, choose any other suitable material(s) for these parts. The support member 154 may be connected to the plate body 151 via adhesive bonding, welding, or other methods; as one alternative, the support member 154 and the plate body 151 may be manufactured as an integral structure.

FIG. 9 is a schematic diagram of the nutritional infusion tubing assembly during the installation process according to an embodiment of this invention. In FIG. 9, the dimensions and positions of structural components such as the peristaltic tube 11 and support member 154 are illustrative.

When an external force is applied to move the support plate 15 away from the roller 241, the peristaltic tube 11 moves with the support plate 15, being stretched and elongated until the support plate 15 reaches a predetermined position, at which point the peristaltic tube 11 is tautly in contact with at least a portion of the roller 241. In this circumstance, the peristaltic tube 11 being in a taut state can be understood as the condition where the tube is just fully extended with only negligible or at most little stretching, well before reaching the point of potential rupture upon further stretching. During this process, the maximum spacing of the peristaltic tube 11 in the first direction can be considered to be gradually decreasing. In the embodiments of this invention, the support member 154 may have a certain degree of elastic deformation capability, meaning that the support member 154 can deform in response to the stretching and elongation of the peristaltic tube 11, thereby adapting to different maximum distances L1 of the peristaltic tube 11 in the first direction. This prevents the support member 154 from obstructing the peristaltic tube 11 during its movement or even rupturing the peristaltic tube 11.

In some embodiments, the support plate 15 may have a centerline O (see FIG. 2), that is perpendicular to the first direction (i.e., in the embodiments of this invention, the centerline O may be perpendicular to the X-axis direction). After the nutritional infusion tubing assembly 10 is installed on the nutritional infusion device 20, the centerline O may pass through the rotational center of the roller 241. The two ends of the peristaltic tube 11 may then be positioned axially symmetric with respect to the centerline O, and the two support members 154 may be axially symmetric with respect to the centerline O, to ensure that the two ends of the peristaltic tube 11 are subjected to uniform force. In other embodiments, the two support members 154 may also be arranged non-axially symmetric, which is not limited in this invention.

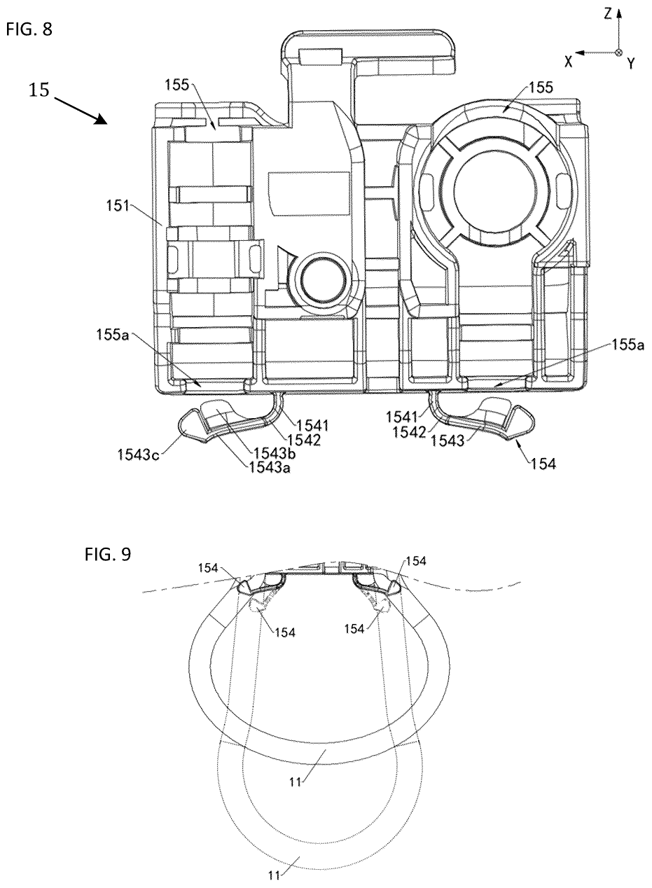

As shown in FIG. 8, for example, the support member 154 may include a connection portion 1541, a bending portion 1542, and a support portion 1543 connected in sequence. The connection portion 1541 is fixedly connected to the plate body 151, the support portion 1543 is spaced apart from the plate body 151, and the support portion 1543 abuts against the annular portion 113. The bending portion 1542 is connected between the connection section 1541 and the support portion 1543, and the bending portion 1542 is curved. By configuring the bending portion 1542 to be curved, the bending resistance performance of the support member 154 is improved, stress-buffering effects are enhanced, and fracture of the support member 154 during deformation is prevented. In other embodiments, the support member 154 may omit the bending portion 1542, and the support member 154 may be approximately straight or have other shapes, abutting between the peristaltic tube 11 and the plate body 151. This invention allows for any preferred shape for the support member 154. By configuring the bending portion 1542 in a curved shape, the bending resistance of the support member 154 can be increased, achieving stress-buffering effects and preventing fracture during deformation. In other embodiments, the support member 154 may omit the bending portion 1542, instead adopting an approximately linear or other geometric configuration where it interfaces between the peristaltic tube 11 and plate body 151. The invention may be implemented with any preferred shape of the support member 154.

In some embodiments, the projecting portion of the support portion 1543 on plate body 151 covers at least part of its corresponding mounting port 155a. Setting the projecting portion of the support portion 1543 on plate body 151 to partially or fully cover its corresponding mounting port 155a, ensures that the length of the support member 154 is sufficient to spread apart the peristaltic tube 11, thereby altering the maximum spacing of the peristaltic tube 11 in the first direction when the nutritional infusion tubing assembly 10 is in its normal state.

In some examples, along the connection direction of the connection portion 1541, bending portion 1542, and support portion 1543, the width of the bending portion 1542 is smaller than the width of the connection portion 1541 and/or the width of the support portion 1543; and/or, a hollowed-out region is provided within the bending portion 1542. By configuring the bending portion 1542 to be narrower or more elastic than the connection portion 1541 and the support portion 1543, the bending portion 1542 becomes more deformable, thereby improving the deformation capability of the support member 154 during the stretching and elongation of the peristaltic tube 11, and enhancing the reliability of support member 154 abutting against peristaltic tube 11.

In some embodiments, the orthogonal projecting portion of the support portion 1543 toward the plate body 151 covers at least part of its corresponding mounting port 155a. Configuring the projecting portion of the support portion 1543 on the plate body 151 to partially or fully cover its corresponding mounting port 155a, ensures that the length of the support member 154 is sufficient to spread the peristaltic tube 11, thereby altering the maximum spacing of the peristaltic tube 11 in the first direction when the nutritional infusion tubing assembly 10 is in its normal state.

As shown in FIGS. 4, 5, and 8, in some embodiments, the support portion 1543 may include a main body section 1543a, a first blocking plate 1543b, and a second blocking plate 1543c. The first blocking plate 1543b and the second blocking plate 1543c are fixed on opposite sides of the main body 1543a, and the peristaltic tube 11 is provided between the first blocking plate 1543b and the second blocking plate 1543c on the main body 1543a. By setting the first blocking plate 1543b and the second blocking plate 1543c, the first blocking plate 1543b and the second blocking plate 1543c can surround at least part of the outer circumference of the peristaltic tube 11, serving to limit and prevent the peristaltic tube 11 from moving in the radial direction and detaching from the support member 154, thereby improving the connection reliability of the support portion 1543 in contact with the peristaltic tube 11. In some embodiments, the support portion 1543 may also not include the first blocking plate 1543b and/or the second blocking plate 1543c.

For example, an orthographic projection of the first blocking plate 1543b toward a surface of the second blocking plate 1543c is offset from the second blocking plate 1543c, which is conducive to reducing the manufacturing difficulty of the support member 154 and facilitates mold manufacturing of the support member 154.

Additionally, when the nutritional infusion tubing assembly 10 is installed in the nutritional infusion device 20, the second blocking plate 1543c may be positioned closer to the bottom surface 151b of the support plate 15 than the first blocking plate 1543b. The second blocking plate 1543c may be configured to prevent the peristaltic tube 11 from disengaging from the mounting groove 155. Besides, by setting the distance between the first blocking plate 1543b and the connection portion 1541 to be smaller than the distance between the second blocking plate 1543c and the connection portion 1541, and/or by making the size of the second blocking plate 1543c larger than the size of the first blocking plate 1543b, the second blocking plate 1543c can surround a larger area around the peristaltic tube 11, which helps the second blocking plate 1543c achieve a better blocking effect.

In some embodiments of this invention, the maximum spacing between the two support members 1543 in the first direction may be within the range of 24 mm to 34 mm. For example, the maximum spacing between the two support members 1543 may be 24 mm, 26 mm, 30 mm, 34 mm, etc. By setting the range of the maximum distance between the two support members 1543 to values at least approximately like these, the length of the support member 154 will be sufficient to support the peristaltic tube 11 while avoiding the support member 154 being too large, which could cause deformation during installation and potential interfere with other structural components of the nutritional infusion device. In other embodiments, the maximum spacing between the two support portions 1543 in the first direction may instead be set according to actual needs.

As shown in FIGS. 2 and 3, the mounting surface 211 may also include structures such as protrusions or grooves to divide the mounting space 23 into several interconnected but differently deep regions, enabling better arrangement and installation of structural components of different sizes within the mounting space 23. In some examples, the mounting space 23 may include, in the Z-axis direction, a first mounting space 23a, a second mounting space 23b, and a third mounting space 23c, which are sequentially connected. In the Y-axis direction, the depth of the first mounting space 23a is greater than that of the second mounting space 23b, and the depth of the third mounting space 23c is greater than that of the second mounting space 23b. For example, in the embodiments of this invention, the mounting surface 211 may also be considered to be divided into a first mounting surface 211a, a second mounting surface 211b, and a third mounting surface 211c. The first mounting surface 211a is located within the first mounting space 23a, the second mounting surface 211b is located within the second mounting space 211b, and the third mounting surface 211c is located within the third mounting space 211c. The first mounting surface 211a, the second mounting surface 211b, and the third mounting surface 211c face in the same direction and are arranged in an offset manner.

In some embodiments of this invention, the roller 241 may be disposed within the first mounting space 23a, and the tube groove 25 may be disposed on the second mounting surface 211b of the second mounting space 23b, as shown in FIGS. 2 and 3. When the nutritional infusion tubing assembly 10 is properly installed, the support plate 15 can be located within the third mounting space 23c, and part of the peristaltic tube 11 can be slotted within the tube groove 25 of the second mounting space 23b, with the peristaltic tube 11 being in contact with at least part of the roller 241 in a straight manner. The first mounting space 23a, second mounting space 23b, and third mounting space 23c may further be divided into multiple sub-mounting spaces with different depths, and this invention does not impose any limitations in this regard. In other embodiments, the dimensional relationships between the first mounting space 23a, second mounting space 23b, and third mounting space 23c may differ from those in the embodiments of this invention, and the mounting space 23 may be divided into more or fewer regions.

In some embodiments, one of the nutritional infusion device 20 and the support plate 15 includes a guide rail, and the other includes a guide channel. To apply an external force to move the support plate 15 away from the roller 241 to a predetermined position, an external force may be applied to move the support plate 15 in a direction perpendicular to the first direction, and the support plate 15 can move along the cooperative trajectory of the guide rail and guide channel to reach the predetermined position. Through the cooperation of the guide rail and the guide channel, the movement trajectory of the support plate 15 can be limited, thereby improving the accuracy of the installation position of the support plate 15.

For example, as shown in FIGS. 3 and 5, in some embodiments of this invention, the nutritional infusion device 20 may include a guide rail 30, and the support plate 15 may include a guide channel 31. During the process of applying an external force to move the support plate 15 away from the roller 241, the guide rail 30 can be slotted into and held within the guide channel 31, and the support plate 15 can move along the extended path of the guide rail 30. In other embodiments, the nutritional infusion device 20 may include the guide channel 31, and the support plate 15 may include the guide rail 30. During the process of applying an external force to move the support plate 15 away from the roller 241, the guide rail 30 can be slotted into the guide channel 31, and the support plate 15 can move along the extended path of the guide channel 31. The guide channel 31 may be located between the two mounting grooves 155. In other embodiments, the number of the guide channels 31 on the support plate 15 may also be two, three, or more, and the guide channel 31 may also be located at other positions on the support plate 15.

For example, the movement directions of the support plate 15 may include a second direction and a third direction, where the first direction, the second direction, and the third direction are mutually perpendicular to any two of them. The guide rails 30 may include a first guide rail 301 and a second guide rail 302, with the first guide rail 301 being closer to the roller 241 than the second guide rail 302. The first guide rail 301 and the second guide rail 302 are arranged in a stepped configuration, and the extension directions of both the first guide rail 301 and the second guide rail 302 are parallel to the second direction.

In some embodiments of this invention, the first guide rail 301 may be installed on the second mounting surface 211b within the second mounting space 23b, and the extension direction of the first guide rail 301 may be parallel to the Z-axis direction. The second guide rail 302 may be installed on the third mounting surface 211c within the third mounting space 23c, and the extension direction of the second guide rail 302 may be parallel to the Z-axis direction. The third mounting space 23c is “sunken” relative to the second mounting space 23b, i.e., the first guide rail 301 and the second guide rail 302 form a stepped configuration, and the first guide rail 301 protrudes relative to the second guide rail 302.

Alternatively, as shown in FIG. 5, the guide channel 31 may include a first guide channel 311 and a second guide channel 312 arranged along the second direction (i.e., the Z-axis direction). When the nutritional infusion tubing assembly 10 is installed on the nutritional infusion device 20, the first guide channel 311 is closer to the roller 241 than the second guide channel 312 (as shown in FIG. 3), the first guide channel 311 and the second guide channel 312 may also be stepped, with the second guide channel 312 protruding relative to the first guide channel 311 and mating with the step between the first guide rail 301 and the second guide rail 302. The movement direction of the support plate 15 is defined by the extension direction of the guide channel 31, which is the same as the movement direction of the support plate 15.

After the nutritional infusion tubing assembly 10 is installed on the nutritional infusion device 20, when an external force is applied to move the support plate 15 in the second direction, the first guide rail 301 is slotted into and held within the first guide channel 311. When an external force is applied to cause the support plate 15 to move in the third direction, the connection point between the first guide channel 311 and the second guide channel 312 abuts against the connection point between the first guide rail 301 and the second guide rail 302.

By configuring the continuous arrangement of guide rails 30 and guide channels 31 to work together, the guide rails 30 provide support and positioning for the support plate 15 during its movement, thereby enhancing the accuracy of the installation position when the nutritional infusion tubing assembly 10 is mounted on the nutritional infusion device 20. Additionally, by configuring the guide channel 31 in a stepped shape, the thickness of the support plate 15 at the second guide channel 312 can be made thinner, which helps reduce manufacturing costs.

In some examples, the guide rail 30 may further include a third guide rail 303, which may be connected between the first guide rail 301 and the second guide rail 302. The third guide rail 303 is perpendicular to the second mounting surface 211b and the third mounting surface 211c. The third guide rail 303 serves as a transition segment, helping to prevent the support plate 15 from sagging during the suspension process. It provides support and positioning for the support plate 15 when it moves in the third direction (i.e., the Y-axis direction).

In other embodiments, the first guide rail 301 and the second guide rail 302 may also be connected in a straight line. In other words, in other embodiments, the guide rails 30 may omit the third guide rail 303. Additionally, when the nutritional infusion device 20 includes the guide channel and the support plate 15 includes the guide rail, the guide rail may also comprise multiple segments of guide rails of different sizes or a single straight guide rail, and may be configured according to actual needs. Other configurations are also possible.

In some embodiments, there is only one guide channel 31 and the two mounting grooves 155 are respectively disposed either side of the guide channel 31, which facilitates the full utilization of the space between the two mounting grooves 155, improves the space utilization rate of the support plate 15, and facilitates the reduction of the size of the support plate 15, achieving miniaturization of the nutritional infusion tubing assembly 10. Additionally, it facilitates the operator's application of force. Furthermore, the two mounting grooves 155 are positioned closer to the edge of the support plate 15 than the guide channel 31, making it easier to install both ends of the peristaltic tube 11 within the mounting grooves 155. The guide channel 31 may be positioned on the centerline O of the support plate 15 or close to the centerline O of the support plate 15.

In some embodiments, as shown in FIGS. 7A and 7B, the top surface 151a of the support plate 15 may be provided with a slope 151c such that the distance between the slope 151c and the bottom surface 151b gradually increases along the direction away from the annular portion 113 of the peristaltic tube 11 (i.e., the Z-axis direction). In some embodiments of this invention, the slope 151c may also be considered to extend upward from a side of the support plate 15 adjacent to the roller 241 toward a side of the support plate 15 away from the roller 241.

By including the slope 151c, it serves as a force-applying point, allowing external force to be applied to move the support plate 15 away from the roller 241. The inclined surface 151c decomposes the operator-applied force into a component parallel to the mounting surface 211 of the nutrient infusion device 20 for pushing/pulling the support plate 15 to elongate the peristaltic tube 11, and a perpendicular component pressing the support plate 15 against the nutritional infusion device 20. This improves installation reliability by ensuring continuous contact between the support plate 15 and the mounting surface 211 and eliminating issues in the prior art where force components during two-handed operation tended to detach the support plate 15 from the nutritional infusion device 20.

As shown in FIGS. 3 and 4, in some embodiments, the nutritional infusion device 20 may further include a fixing component 26, which may be fixed to the housing 21 and located on a side of the tube groove 25 back away from the roller 241. For example, the fixing component 26 may include structures such as through holes. The support plate 15 of the nutritional infusion tubing assembly 10 may include a protrusion 152, which is fixed to the top of the plate body 151. The protrusion 152 may be slotted into the fixing component 26 to fix the relative position between the support plate 15 and the fixing component 26, thereby achieving positioning of the relative position between the nutritional infusion tubing assembly 10 and the nutritional infusion device 20. The protrusion 152 and the plate body 151 may be an integral structure or an integrated structure formed by welding, adhesive bonding, or other methods. The protrusion 152 may be made of materials such as acrylonitrile-butadiene-styrene (ABS) resin, which has high strength and toughness. This invention does not specify the materials for the protrusion 152 and the plate body 151. In other embodiments, the protrusion 152 may be located at other positions on the plate body 151, or the nutritional infusion tubing assembly 10 may not include the protrusion 152. The support plate 15 of the nutritional infusion tubing assembly 10 may cooperate with the fixing component 26 through other structures to achieve positioning.

It can be understood that during the process of slotting the protrusion 152 into the fixing component 26, the protrusion 152 may be compressed and deformed due to its elasticity. When the support plate 15 is properly installed, the protrusion 152 can return to its original state and snap into place within the fixing component 26. Alternatively, the protrusion 152 may remain in a compressed state, and the deformation recovery force of the protrusion 152 can be used to push against the fixing component 26, thereby enhancing the tightness of the connection between the fixing component 26 and the protrusion 152.

For example, along the direction from the top surface 151a of the support plate 15 toward the bottom surface 151b of the support plate 15, the distance between the plate body 151 and the surface of the protrusion 152 back away from the plate body 151 gradually decreases. It can be understood that along the direction from the top surface 151a of the support plate 15 toward the bottom surface 151b of the support plate 15, i.e., along the extension direction of the third guide rail 303, the distance between the plate body 151 and the surface of the protrusion 152 back away from the plate body 151 gradually decreases, which facilitates squeezing the protrusion 152 into the fixing component 26 during the movement of the support plate 15 along the third guide rail 303. In some examples, the nutritional infusion tubing assembly 10 may further include a pressing member 156, which is elastically connected between the protrusion 152 and the plate body 151 to provide deformation space for the protrusion 152 during its compression into the fixing component 26. For example, the pressing member 156 may be approximately bow-shaped, or have a shape resembling a partial bow-shaped structure, or the pressing member 156 may also have hollowed-out regions internally to provide a larger deformation space for the protrusion 152. Additionally, when removing the nutritional infusion tubing assembly 10 from the nutritional infusion device 20, pressing the pressing member 156 can also cause the protrusion 152 to disengage from the fixing component 26.

In some embodiments, the nutritional infusion device 20 may further include a locking member 27, the support plate 15 may be provided with a locking hole 153, and the locking member 27 may be inserted into the locking hole 153 to achieve a limiting effect on the nutritional infusion tubing assembly 10. The locking member 27 may be slidably or rotatably connected to the housing 21. By rotating or moving the locking member 27, the support plate 15 can be positioned in other directions. For example, in some embodiments of this invention, as shown in FIG. 2, the locking hole 153 is provided with a first blocking plate and a second blocking plate spaced apart along the Z-axis direction. At the initial position, the locking member 27 can extend along the X-axis direction. In this circumstance, the locking member 27 can be slotted in the space between the first blocking plate and the second blocking plate, thereby preventing the support plate 15 from moving relative to the housing 21 along the Z-axis direction. By rotating the locking member 27 to change its position, it can also extend along the Z-axis and come into contact with the space between the first and second blocking plates, thereby preventing the support plate 15 from moving relative to the housing 21 along the Y-axis direction. This invention does not require any particular connection method between the locking member 27 and the housing 21, nor the relative position between the locking member 27 and the locking hole 153. In other embodiments, the nutritional infusion system 1 may for example omit the locking member 27 and the locking hole 153 altogether.

In some embodiments, the nutritional infusion tubing assembly 10 may also include an in-place identification element 16 (as shown in FIGS. 6A and 6B), and the nutritional infusion device 20 may include a detection element (not shown in the figures). The detection element may be a Hall sensor. Utilizing the Hall effect principle, the Hall sensor can feed back the magnitude of the surrounding magnetic field through the output Hall voltage value. The voltage value output by the Hall sensor when the nutritional infusion tubing assembly 10 is installed in the nutritional infusion device 20 differs from the voltage value output by the Hall sensor when the nutritional infusion tubing assembly 10 is not installed, thereby enabling detection of the installation status of the nutritional infusion tubing assembly 10. Additionally, the Hall sensor may also be configured to identify the category of the nutritional infusion tubing assembly 10 installed on the nutritional infusion device 20. For example, when the valve structure inside the nutritional infusion tubing assembly 10 is a two-way valve, the “N” pole of the in-place identification element 16 inside the nutritional infusion tubing assembly 10 may be oriented toward the Hall sensor. Similarly, when the valve structure inside the nutritional infusion tubing assembly 10 is a three-way valve, the “S” pole of the in-place identification element inside the nutritional infusion tubing assembly 10 may be oriented toward the Hall sensor. Thus, when different nutritional infusion tubing assemblies 10 are installed on the same nutritional infusion device 20, the Hall voltage values detected by the Hall sensor will also differ, thereby identifying different types of nutritional infusion tubing assemblies 10. In other embodiments, the detection of whether the nutritional infusion tubing assembly 10 is installed on the nutritional infusion device 20 may also be achieved by installing a tactile switch, photoelectric sensor, etc., within the nutritional infusion device 20. For example, the locking hole 153 and the in-place identification element 16 may be located between the guide channel 31 and the mounting groove 155 for mounting the output tube connector 13. The size of the input tube connector 12 is generally larger than that of the output tube connector 13. By positioning the locking hole 153 and the in-place identification element 16 close to the output tube connector 13, the space within the support plate 15 can be fully utilized, facilitating the miniaturization of the support plate 15 and the nutritional infusion tubing assembly 10, and enabling the operator to perform operations with one hand.

In some embodiments, the nutritional infusion device 20 may further include a switching member 28. The valve structure of the nutritional infusion tubing assembly 10 may be provided with a direction-changing groove 121, and the switching member 28 may be provided within the direction-changing groove 121 of the valve structure to drive the rotation of the valve core of the valve structure, thereby controlling the switching of the valve core between different positions, enabling the opening, closing, and switching of the fluid pathway within the nutritional infusion tubing assembly 10, and reducing the difficulty for the operator in using the nutritional infusion system 1.

In the preceding embodiment, the nutritional infusion tubing assembly 10 is designed correspondingly based on an exemplary configuration where the support member 154 is disposed within the support plate 15. In other embodiments, the nutritional infusion tubing assembly 10 may omit the support member 154, and the outward expansion force may be provided by modifying other structures within the nutritional infusion tubing assembly 10, such as the input tube connector 12 and/or the output tube connector 13, to prevent the output section 112 of the peristaltic tube 11 from approaching the input section 111 of the peristaltic tube 11 in the first direction.

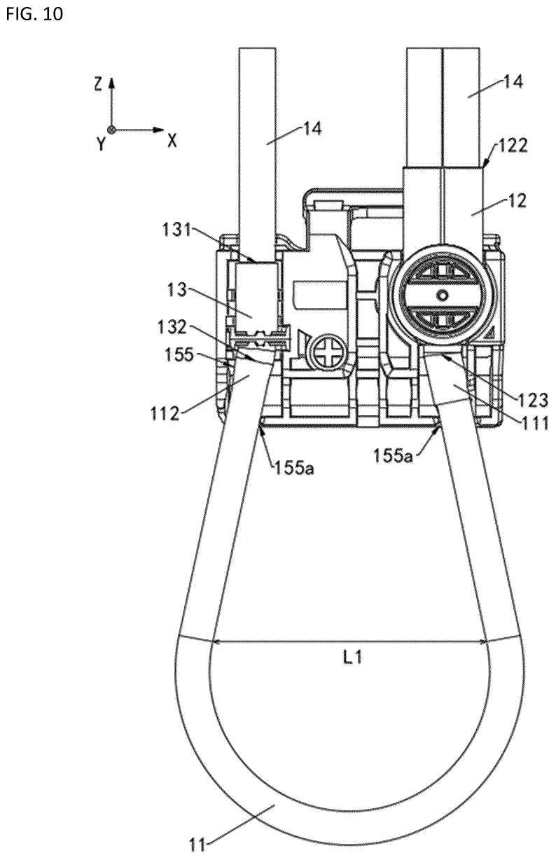

For example, FIG. 10 is a structural schematic diagram of another nutritional infusion tubing assembly according to an embodiment of this invention. In some embodiments, the input tube connector 12 may include a first end 122 and a second end 123. The first end 122 is connected to at least one infusion tube 14 and the second end 123 is connected to the peristaltic tube 11, and the first end 122 is bent relative to the second end 123. In some embodiments, the output tube connector 13 may include a third end 131 and a fourth end 132. The third end 131 is connected to another infusion tube 14, and the fourth end 132 is connected to the peristaltic tube 11, and the third end 131 is bent relative to the fourth end 132. By altering the shape of the input tube connector 12 and/or the shape of the output tube connector 13, it is also possible to prevent the output section 112 of the peristaltic tube 11 from approaching the input section 111 of the peristaltic tube 11 in the first direction, so that the maximum distance L1 between the peristaltic tube 11 and the nutritional infusion tubing assembly 10 in the normal state in the first direction is greater than or equal to the maximum distance L2 between any two points of the roller 241 (as shown in FIG. 2).

It can be understood that, as shown in FIG. 10, the second end 123 is located within the mounting groove 155. In some embodiments, the second end 123 may also be flush with or protrude from the mounting port 155a; and/or, the fourth end 132 may be installed on another mounting port 155a, flush with or extending beyond the other mounting port 155a. The closer the second end 123 is to the roller 241, the stronger the support force of the input tube connector 12 on the peristaltic tube 11 will be, and the greater the influence on its shape. Similarly, the closer the fourth end 132 is to the roller 241, the stronger the support force of the output tube connector 13 on the peristaltic tube 11 will be, and the greater the influence on its shape. Making the second end 123 and/or the fourth end 132 to be flush with or protrude from the mounting port 155a, is advantageous because it ensures that the maximum spacing L1 of the peristaltic tube 11 is greater than the maximum spacing L2 between any two points of the roller 241 when the nutritional infusion tubing assembly 10 is in its normal state.

In other embodiments, the orientation of at least a portion of the section of the peristaltic tube 11 between the input tube connector 12 and/or the output tube connector 13 and the mounting port 155a may be altered by increasing the number of grooves on the support plate 15 or modifying the spacing on the support plate 15. For example, a transverse tube groove (not shown in the figures) can be provided, with the extension direction of the transverse tube groove intersecting or expending perpendicular to the extension direction of the guide channel 31. The groove walls of the transverse tube groove can then provide an outward expansion force, which increases the distance between the two starting points of the annular portion 113 along the direction perpendicular to the guide channel 31 (i.e., the X-axis direction). This in turn allows the peristaltic tube 11 to be easily looped around the roller 241 despite its smaller size, which simplifies the installation process of the nutritional infusion tubing assembly 10, and makes it easier to operate. This invention does not require this configuration, however.

Described above are only some of the embodiments and implementations of this invention, and the scope of protection of this invention is not limited thereto. Any skilled person skilled in the art can easily think of changes or modifications within the scope of what is disclosed herein, which shall fall within the scope of protection of this invention. Therefore, the scope of protection of this invention shall be subject to the scope of protection of the claims.

1. A nutritional infusion tubing assembly, comprising:
a peristaltic tube,
an input tube connector,
an output tube connector,
infusion tubes, and
a support plate;
wherein:
the support plate is provided with a guide channel and two mounting grooves, with the two mounting grooves located on opposite sides of the guide channel;
the guide channel is configured to guide movement of the support plate;
an input end of the peristaltic tube is connected to one end of the input tube connector, another end of the input tube connector is connected to at least one of the infusion tubes, and the input tube connector is located in one of the mounting grooves;
an output end of the peristaltic tube is connected to one end of the output tube connector, another end of the output tube connector is connected to another one of the infusion tubes, and the output tube connector is located within the other mounting groove;
between the input end and the output end, the peristaltic tube further comprises an annular portion suspended relative to one of: the support plate and an independent expansion member;
a section of the peristaltic tube between the input end and a first starting point of the adjacent annular portion is subjected to an outward expansion force from one of: the support plate, the input tube connector, and the independent expansion member;
a section of the peristaltic tube between the output end and a second starting point of the adjacent annular portion is subjected to the outward expansion force from one of: the support plate, the output tube connector and the independent expansion member;
the outward expansion force has a component along a direction perpendicular to an extension direction of the guide channel, thereby increasing a distance between the first and second starting points of the annular portion along the direction perpendicular to the extension direction of the guide channel.

2. The nutritional infusion tubing assembly of claim 1, wherein the guide channel is positioned on a centerline of the support plate.

3. The nutritional infusion tubing assembly of claim 1, further comprising one of: a locking hole and an in-place identification element, located between the guide channel and the mounting groove for installing the output tube connector.

4. The nutritional infusion tubing assembly of claim 1, wherein
the support plate is provided with a top surface and a bottom surface, which are located on opposite sides of the support plate;
the top surface is provided with a slope, and
in a direction extending away from the annular portion of the peristaltic tube, a distance between the slope and the bottom surface gradually increases.

5. The nutritional infusion tubing assembly of claim 1, wherein
the support plate is provided with a top surface and a bottom surface, which are located on opposite sides of the support plate, and
the mounting grooves are provided on the bottom surface;
the support plate comprises a plate body and a protrusion, wherein the protrusion is provided at a top end of the plate body, and along a direction from a top surface of the support plate toward a bottom surface of the support plate, a distance between the plate body and a surface of the protrusion back away from the plate body gradually decreases.

6. The nutritional infusion tubing assembly of claim 1, wherein the support plate comprises a plate body and two support members;
the two mounting grooves and the guide channel are provided on the plate body, the two support members are spaced apart on the same side of the plate body or provided on the corresponding opposite sides of the plate body, respectively;
one of the support members abuts the starting point in one end of the annular portion, and the other support member abuts the starting point of the other end in the annular portion, thereby increasing the distance between the two starting points of the annular portion in the direction perpendicular to the guide channel.

7. The nutritional infusion tubing assembly of claim 1, wherein an end of one of the input tube connectors and the output tube connector is bent relative to an end for connecting to the peristaltic tube.

8. The nutritional infusion tubing assembly of claim 1, wherein
the support plate comprises two mounting ports and a transverse tube groove, wherein
the mounting ports are provided for the peristaltic tube to pass through and connect to the input tube connector and the output tube connector, respectively, and
an extension direction of the transverse tube groove is at least approximately perpendicular to the extension direction of the guide channel;
wherein the section of the peristaltic tube from the input tube connector or the output tube connector to the corresponding mounting port is at least partially arranged along the transverse tube groove.

9. The nutritional infusion tubing assembly of claim 1, wherein the maximum spacing of the annular portion in the extension direction of the guide channel is at least 33 mm.

10. The nutritional infusion tubing assembly of claim 1, wherein two ends of the annular portion are axially symmetric about a centerline of the support plate.

11. A nutritional infusion tubing assembly, comprising
a peristaltic tube,
an input tube connector,
an output tube connector, infusion tubes, and
a support plate,
wherein:
the input tube connector and the output tube connector are both fixed to the support plate, one end of the input tube connector is connected to an input end of the peristaltic tube, and another end of the input tube connector is connected to at least one infusion tube;
one end of the output tube connector is connected to an output end of the peristaltic tube, and another end of the output tube connector is connected to another infusion tube;
the support plate comprises a plate body and two support members,
the input tube connector and the output tube connector are both fixed to the plate body,
the two support members are spaced apart on the plate body;
one support member abuts the input end of the peristaltic tube, and the other support member abuts the output end of the peristaltic tube, to prevent at least part of the input end of the peristaltic tube and at least part of the output end of the peristaltic tube from approaching each other in a direction toward a centerline of the plate body.

12. The nutritional infusion tubing assembly of claim 11, wherein the two support members are located between the input end of the peristaltic tube and the output end of the peristaltic tube.

13. The nutritional infusion tubing assembly of claim 11, wherein
the support member comprises a connection portion and a support portion,
the connection portion is fixedly connected to the plate body, and
the support portion is spaced apart from the plate body and abuts the input end or the output end of the peristaltic tube.

14. The nutritional infusion tubing assembly of claim 13, further comprising a bending portion that connects the connecting portion and the support portion, wherein the bending portion is curved; and/or, along a connection direction of the connection portion, the bending portion, and the support portion, the width of the bending portion is smaller than the width of the connection portion and the width of the support portion.

15. The nutritional infusion tubing assembly of claim 13, wherein
the plate body is provided with a mounting port,
the peristaltic tube is mounted in the mounting port, and
an orthogonal projection of the support portion toward the plate body covers at least part of the mounting port.

16. The nutritional infusion tubing assembly of claim 13, wherein the maximum distance between the two support portions is within the range of 24 mm to 34 mm.

17. The nutritional infusion tubing assembly of claim 13, wherein
the support portion comprises a main body, a first blocking plate, and a second blocking plate,
the first blocking plate and the second blocking plate are fixed on opposite sides of the main body, and
the peristaltic tube is located on the main body between the first blocking plate and the second blocking plate.

18. The nutritional infusion tubing assembly of claim 17, wherein
the plate body is provided with a top surface and a bottom surface,
the peristaltic tube is closer to the bottom surface of the support plate than to the top surface of the support plate,
the second blocking plate is positioned closer to the bottom surface than the first blocking plate;
an orthographic projection of the first blocking plate toward a surface of the second blocking plate is offset from the second blocking plate;
a distance between the first blocking plate and the connection portion is less than a distance between the second blocking plate and the connection portion, and/or a size of the second blocking plate is larger than a size of the first blocking plate.

19. The nutritional infusion tubing assembly of claim 1, wherein two ends of the peristaltic tube are axially symmetric about the centerline of the plate body, and the two support members are axially symmetric about the centerline of the plate body.

20. A nutritional infusion tubing assembly, comprising:
a peristaltic tube,
an input tube connector,
an output tube connector,
infusion tubes, and
a support plate;
wherein
the support plate is provided with a guide channel and two mounting grooves, with the two mounting grooves located on opposite sides of the guide channel;
the guide channel is configured to guide movement of the support plate;
an input end of the peristaltic tube is connected to one end of the input tube connector,
another end of the input tube connector is connected to at least one of the infusion tubes, and the input tube connector is located in one of the mounting grooves;
an output end of the peristaltic tube is connected to one end of the output tube connector,
another end of the output tube connector is connected to another one of the infusion tubes, and the output tube connector is located within the other mounting groove;
between the input end and the output end, the peristaltic tube has an annular portion suspended relative to the support plate or an independent expansion member;
the support plate comprises a plate body and two support members, with the two support members spaced apart on a bottom side of the plate body and extending toward the annular portion; and
one of the support members abuts the starting point in one end of the annular portion, and the other support member abuts the starting point of the other end in the annular portion, thereby increasing the distance between the two starting points of the annular portion along the direction perpendicular to the guide channel.
